(19) United States
(12) Patent Application Publication
(10) Pub. No.: US 2015/0029492 A1
(43) Pub. Date: Jan. 29, 2015

(54) MEASUREMENT OF TOTAL HEMOGLOBIN IN WHOLE BLOOD

(71) Applicant: MEC DYNAMICS CORPORATION, San Jose, CA (US)

(72) Inventors: Emmanuel Mpock, Salida, CA (US); Wilma Mangan, Santa Clara, CA (US)

(21) Appl. No.: 14/391,376

(22) PCT Filed: Apr. 9, 2013
(86) PCT No.: PCT/US2013/035849

§ 371 Date: Oct. 8, 2014

Related U.S. Application Data
(60) Provisional application No. 61/686,670, filed on Apr. 9, 2012.

(51) Int. Cl. G01N 33/72; G01N 21/59

(52) U.S. Cl. CPC G01N 33/721; G01N 21/59; G01N 2201/061
USPC 356/42; 356/40

(57) ABSTRACT

A method for determining total hemoglobin concentration in a blood sample comprising spectrophotometric analysis of a blood sample at two wavelengths and determining a ratio of the detected radiation at a first wavelength to the detected radiation at a second wavelength; and determining a concentration of total hemoglobin in the blood sample based on the ratio.

### RELATED APPLICATIONS

This application claims the benefit of the earlier filing date of U.S. Provisional Application 61/686,670 filed Apr. 9, 2012 which is hereby incorporated by reference for all purposes.

### FIELD

The disclosure relates the direct determination of total hemoglobin concentration in whole blood without the need for transforming the hemoglobin through secondary reactions.

### BACKGROUND

Traditional total hemoglobin measurements have generally been done indirectly, requiring significant sample quantities from a venous draw, extensive sample preparation and the use of toxic and dangerous chemicals. The assay reagents are typically used to lyse the blood cells to release the hemoglobin molecules to which accompanying molecules react with and bind unto the hemoglobin molecules to form a stable compound that is optically detectable, and whose concentrations are proportional to the amount of hemoglobin in the sample. The need for reagents adds to the cost of the assay and makes it less accessible in the developing world. Certain reagents also require specific storage conditions which makes them less useful in areas where climate control is rare. Thus there is a need for a method to determine total hemoglobin content in modified or unmodified forms even in very small blood samples.

### BRIEF DESCRIPTION OF THE DRAWINGS

FIG. 1 illustrates sample holders according to two embodiments.

FIG. 2 illustrates measurements along a capillary according to one embodiment.

FIG. 3 illustrates a calibration curve according to one embodiment.

FIG. 4 illustrates linearity of the response.

### DETAILED DESCRIPTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

All publications, patents and patent applications cited herein, whether supra or infra, are hereby incorporated by reference in their entirety.

Described herein is a convenient and inexpensive method for quantifying total hemoglobin directly and rapidly in a system designed for use at the point of care using a very small blood sample. The sample requires no addition of reagents to accomplish the measurement. The sample is exposed to light at a plurality of wavelengths. Absorbance, transmission or reflectance measurements are made and the ratio of the measurements at two wavelengths indicate the amount of total hemoglobin.

### Sample Preparation

A small amount of blood is drawn into a sample holder. Only a few drops are needed. The sample holder can be any holder normally used to collect blood samples provided that the holder material allows transmission of the wavelengths of light used for analysis. Example sample holders include glass capillaries (open on both ends) as well as test strips incorporating a capillary channel that is open to the atmosphere at both ends of the channel. When one end of the capillary is touched to a drop of blood, the blood is drawn into the channel. The sample holder may be uncoated and need not contain any reagents. No reagents (such as lysing agents) need to be added to the sample. It should be noted however that the disclosed method can be used with samples where a lysing agent has been added to the blood sample. The sample is then analyzed spectrophotometrically.

Example sample holders of various shapes are shown in FIG. 1. The substrates holding the capillary channel can be cylindrical, rectangular or polymorphic.

### Sample Analysis

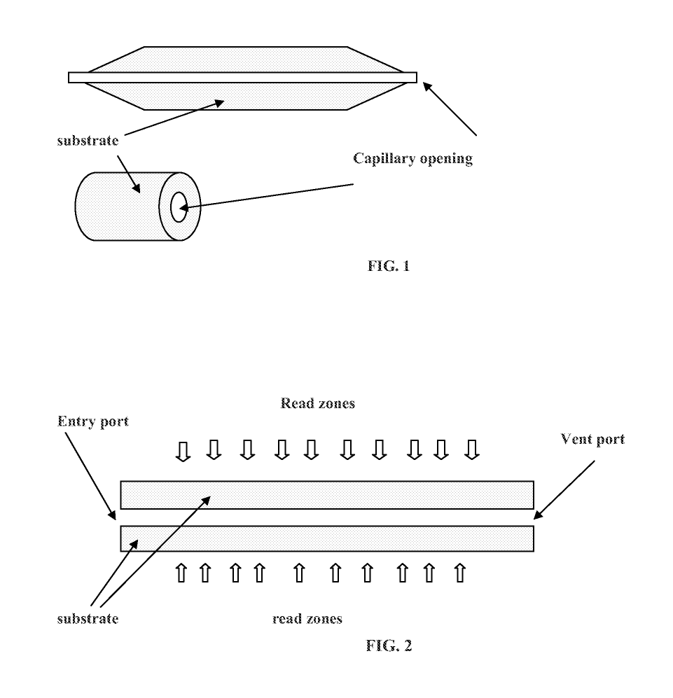

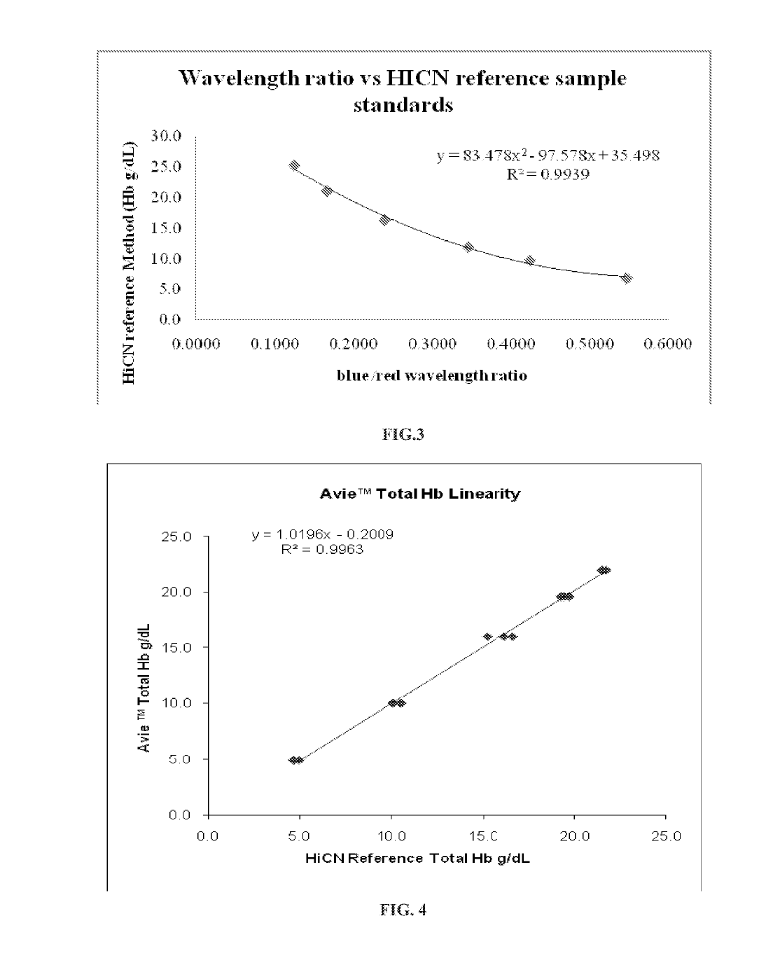

The sample is exposed to light at at least two wavelengths. The selected wavelengths should be sufficiently separated on the spectrum but this does not mean that the edges of the ranges of the two wavelengths cannot overlap. While not wishing to be bound by any particular theory, it is believed that the use of multiple wavelengths avoids error due to hemoglobin variants and side reactions. In one embodiment, the sample is exposed to both blue light as well as red light. In some embodiments the blue light has wavelengths spanning 390 to 520 nm, or 390 to 495 nm, or any subrange of any of the foregoing. In some embodiments, the blue light has a wavelength of between 390 nm and 520 nm or a plurality of wavelengths selected from wavelengths of 390 nm to 520 nm. In some embodiments, the red light has wavelengths spanning 500 to 700 nm, or 570 to 750 nm, or 620 to 750 nm or any subrange of any of the foregoing. In some embodiments, the red light has a wavelength of between 500 nm and 700 nm or a plurality of wavelengths selected from wavelengths of 500 nm to 700 nm. As shown in FIG. 2, the measurements can be made along any portion of the capillary channel provided that the substrate at that portion allows transmission of the wavelengths used for analysis.

The reflectance of the light off of the sample is detected or alternatively, the transmission of light through the sample of light is detected. In yet another alternative, the absorbance of light by the sample is determined. Standard spectrophotometric techniques can be used to make the spectrophotometric measurements. For ease of detection of the multiple wavelengths, the reflected or transmitted light is detected by a detector array such as a charge coupled device (CCD) or photodiode array. The sample is exposed to light for an extended period of time during which several measurements are taken. The sample may be exposed from 5-20 seconds, from 8-15 seconds or about 10 seconds. Measurements are taken every second, every half second or every quarter second. In some embodiments, the measurements are taken at several positions along the capillary channel.

The measurements for each color (or wavelength) of light are averaged and then the ratio is taken of the average of one color (wavelength) to the average of the other color (wavelength). If measurements are taken at multiple positions along the capillary channel, all measurements, regardless of position, for each color (wavelength) are averaged. For example if the measurements are transmission measurements and the light is blue light and red light, the ratio is determined as follows: (average of all transmission measurements of blue light)/(average of all transmission measurements of red light). This ratio correlates to the total hemoglobin concentration in the sample. After creation of a calibration curve, the ratio of optical measurements for a sample is used to determine the total hemoglobin concentration for the sample.

When larger samples are available, this method can be used with larger sample holders and spectrophotometers that hold larger volumes.

In some embodiments, the sample is exposed to many wavelengths of light and the two wavelengths used for analysis are those that are measured spectrophotometrically.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Determination of Calibration Curve

Haemoglobincyanide (HiCN) references were prepared at 6.8, 9.7, 11.9, 16.3, 21.0 and 25.2 g/dL. The standards were prepared and the calibration curve calculated using the standard World Health Organization (WHO) method (“Haemoglobinometry” Chap 7 in the Blood and Safety and Clinical Technology, Guidelines on Standard Operating Procedures for Haematology published by WHO). The transmission of red light (from 500 to 700 nm) and blue light (from 390 to 520 nm) through the sample was measured. For each sample the transmission of each color was measured 20 times at ½ second intervals. The ratio of blue to red was determined for each measurement and then the average ratio was determined. The last 5 measurements for each sample are shown below in Table 1. Spectrophotometric analysis was performed by the Avie™ Alc available from MEC Dynamics in San Jose, Calif.

TABLE 1

| | | Avg(with sample) WB | | | |
|---|---|---|---|---|---|
| | | Red | Blue | | |
| Sample | Ref. | trans- | trans- | | Avg |
| run | HiCN | mission | mission | blu/red | blue/red |
| 1 | 6.8 | 56487 | 30843 | 0.5460 | 0.5464 |
| | | 56437 | 30858 | 0.5468 | |
| | | 56737 | 30977 | 0.5460 | |
| | | 56437 | 30858 | 0.5468 | |
| | | 56487 | 30873 | 0.5466 | |
| 2 | 9.7 | 55363 | 23460 | 0.4237 | 0.4239 |
| | | 55124 | 23366 | 0.4239 | |
| | | 55172 | 23383 | 0.4238 | |
| | | 55124 | 23374 | 0.4240 | |
| | | 55124 | 23374 | 0.4240 | |
| 3 | 11.9 | 53826 | 18615 | 0.3458 | 0.3458 |
| | | 54054 | 18686 | 0.3457 | |
| | | 53826 | 18615 | 0.3458 | |
| | | 53826 | 18610 | 0.3457 | |
| | | 53826 | 18615 | 0.3458 | |
| 4 | 16.3 | 51821 | 12427 | 0.2398 | 0.2398 |
| | | 51821 | 12424 | 0.2397 | |
| | | 52032 | 12473 | 0.2397 | |
| | | 51779 | 12427 | 0.2400 | |
| | | 52032 | 12478 | 0.2398 | |
| 5 | 21.0 | 50393 | 8396 | 0.1666 | 0.1666 |
| | | 50354 | 8396 | 0.1667 | |
| | | 50393 | 8397 | 0.1666 | |
| | | 50393 | 8395 | 0.1666 | |
| 6 | | 50393 | 8396 | 0.1666 | |
| | 25.2 | 49459 | 6188 | 0.1251 | 0.1251 |
| | | 49268 | 6162 | 0.1251 | |
| | | 49230 | 6164 | 0.1252 | |
| | | 49230 | 6163 | 0.1252 | |
| | | 49459 | 6189 | 0.1251 | |

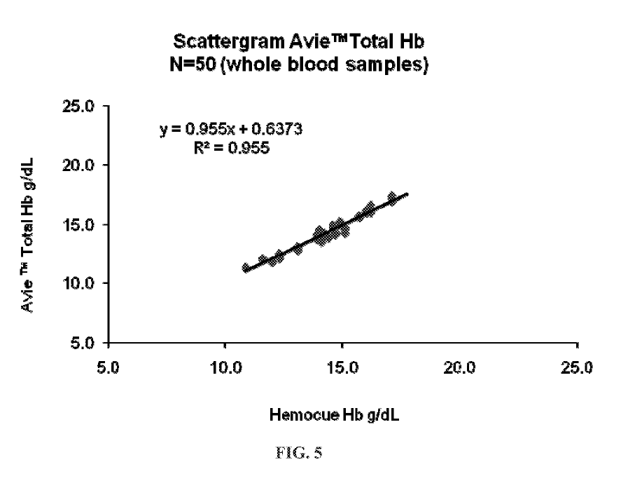

The resulting calibration curve is shown as FIG. 3.

### Example 2

### Linearity of Response

HiCN references from 5 to 23 g/dL were prepared by serial dilution and their concentration determined using the measurement procedure described in Example 1. FIG. 4 illustrates the linearity:

### Example 3

### Accuracy of Response

Fifty whole blood samples were tested using both the procedure in Example 1 as well as using a Hemocue™ Hb 201 available from HemoCue AB of Angelholm Sweden. FIG. 5 illustrates excellent accuracy for the disclosed method as compared to HemoCue™ Hb 201.

### Example 4

### Precision of Response

To assess the precision of the disclosed method, samples having low, mid and high hemoglobin concentrations were tested 20 times each using the method in Example 1. The results in Table 2 illustrate the precision of the disclosed method.

| | Low Hb | Mid Hb | High Hb |
|---|---|---|---|
| Replicate | Total Hb g/dL | Total Hb g/dL | Total Hb g/dL |
| 1 | 6.3 | 13.9 | 18.6 |
| 2 | 6.1 | 13.8 | 18.5 |
| 3 | 6.0 | 13.5 | 18.6 |
| 4 | 6.2 | 13.8 | 18.3 |
| 5 | 6.5 | 13.8 | 18.6 |
| 6 | 6.3 | 13.7 | 18.7 |
| 7 | 6.1 | 14.1 | 18.6 |
| 8 | 6.4 | 14.3 | 18.6 |
| 9 | 6.0 | 14.2 | 18.7 |
| 10 | 6.3 | 14.0 | 18.6 |
| 11 | 6.2 | 13.9 | 18.7 |
| 12 | 6.2 | 14.2 | 18.5 |
| 13 | 6.3 | 14.5 | 18.7 |
| 14 | 6.2 | 14.1 | 18.9 |
| 15 | 6.1 | 13.9 | 18.7 |
| 16 | 6.1 | 14.0 | 18.8 |
| 17 | 6.2 | 14.2 | 19.1 |
| 18 | 5.8 | 14.1 | 18.7 |
| 19 | 6.0 | 14.0 | 18.9 |
| 20 | 6.1 | 14.2 | 18.9 |
| Avg | 6.2 | 14.0 | 18.7 |
| SD | 0.2 | 0.2 | 0.2 |
| % CV | 2.6 | 1.7 | 0.9 |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A method for determining total hemoglobin concentration in a blood sample comprising:
directing radiation having a first wavelength and radiation having a second wavelength through a blood sample;
detecting the radiation at the first wavelength and the radiation at the second wavelength after passing through the blood sample;
determining a ratio of the detected radiation at the first wavelength to the detected radiation at the second wavelength; and
determining a concentration of total hemoglobin in the blood sample based on the ratio.

2. The method of claim 1 wherein the radiation having a first wavelength is blue light.

3. The method of claim 1 wherein the radiation having a first wavelength comprises radiation having wavelengths of 390 nm to 520 nm.

4. The method of claim 1 wherein the radiation having a first wavelength comprises radiation having a wavelength of between 390 nm and 520 nm.

5. The method of claim 1 wherein the radiation having a first wavelength comprises radiation having a plurality of wavelengths selected from wavelengths of 390 nm to 520 nm.

6. The method of claim 1 wherein the radiation having a second wavelength is red light.

7. The method of claim 1 wherein the radiation having a second wavelength comprises radiation having wavelengths of 500 nm to 700 nm.

8. The method of claim 1 wherein the radiation having a second wavelength comprises radiation having a wavelength of between 500 nm and 700 nm.

9. The method of claim 1 wherein the radiation having a second wavelength comprises radiation having a plurality of wavelengths selected from wavelengths of 500 nm to 700 nm.

10. The method of claim 1 wherein:
detecting the radiation at the first wavelength and the radiation at the second wavelength comprises detecting a plurality of signals generated by the radiation at the first wavelength and detecting a second plurality of signals generated by the radiation at the second wavelength; and
determining the ratio of the detected radiation comprises determining an average of the plurality of signals and determining a second average of the second plurality of signals and determining a ratio of the average to the second average.

11. The method of claim 1 wherein determining a concentration of total hemoglobin in the blood sample based on the ratio comprises preparing a calibration curve of standard total hemoglobin solutions and determining the concentration of total hemoglobin in the blood sample based on the calibration curve.

12. The method of claim 1 wherein the blood sample has not been contacted by a lysing reagent.

13. The method of claim 1 wherein red blood cells in the blood sample remain whole.
